(19) United States
(12) Patent Application Publication
(10) Pub. No.: US 2002/0142006 A1
(43) Pub. Date: Oct. 3, 2002

(54) Chimeric nontoxic mutants of enterotoxins as mucosal adjuvants for cell-mediated or humoral immunity

(76) Inventors: Jerry R. McGhee, Hoover, AL (US); Hiroshi Kiyono, Ashiya (JP); Yoshifumi Takeda, Tokyo (JP); Mari Ohmura, Niigata (JP)

Correspondence Address: Glenna Hendricks, Esq., P.O. Box 2509, Fairfax, VA, 22031-2509, US

(21) Appl. No.: 09/809,033

(22) Filed: Mar. 16, 2001

Related U.S. Application Data
(60) Provisional application No. 60/109,058, filed on Nov. 19, 1998.

(51) Int. Cl. A61K 39/108; C07K 14/28; C07K 14/245

(52) U.S. Cl. 424/241.1; 530/350

(57) ABSTRACT

Customized chimeric mutants having a mutated A chain from a first toxin and a b chain from a second toxin provide customized constructs which can be directed to selectively provide cell-mediated immune response or humoral immune response.

### This application takes priority from Provisional Patent Application 60/109,058, filed Mar. 17, 2000.

The research underlying this invention was supported by Grant No. No. NO1 AI65299 from the National Institutes of Health. Hence, certain rights are reserved for the United States Government.

### FIELD OF THE INVENTION

This invention relates to enhancement and customization of immune response using chimeric toxins as adjuvants, said chimera containing a mutated A subunit of one toxin attached to a B subunit of a second enterotoxin. The B subunit is chosen specifically for its binding site.

### BACKGROUND OF THE INVENTION

Cholera toxin (CT) is an exotoxin produced by Vibrio cholerae and has 80% structural homology with the exotoxin produced by enterotoxigenic Escherichia coli (ETEC) termed labile toxin (LT). Both CT and LT are enterotoxins, e.g., they induce a watery diarrhea in humans with cholera or ETEC infections, and they both exhibit an AB-type structure. Thus, each molecule has one A subunit which is an ADP-ribosyl transferase enzyme that binds to AND (nicotinamide adenosine diphosphate) and catalyzes ADP ribosylation of the G protein Gsα. (See Spangler, “Structure and Function of Cholera Toxin and the Related Escherichia coli Heat-labile Enterotoxin”, Microbiol. Rev 56:622-647 (1992).) This Gsai binds GTP and activates adenyl cyclase with subsequent elevation of cyclic AMP. When this occurs in the gastroin-testinal tract, the affected epithelial cells secrete water and chloride ions causing a watery diarrhea in the host organism. (See Gill, et al, “The Mechanism of Action of Cholera Toxin in Pigeon Erythrocyte Lysates”, J. Biol. Chem. 250:6424-6432 (1975).)

Both CT and LT also have a B subunit pentamer linked to the toxic Al subunit and each B subunit is an identical 11.6 kDa peptide. (See Van Heyningen, “Cholera Toxin: Interaction of Subunits with Ganglioside GM1”, Science 183:656-657 (1974).) Thus, a 58 kDa pentameric B subunit is co-valently linked to the 28 kDa enzymatic A1 subunit via A2, a 5 kDa helical peptide, whose main function appears to be as a linker joining A1 with a pentameric CT-B or LT-B. The CT-B differs from LT-B in that the former only binds to GM1 ganglioside on eukaryotic cells, while LT-B binds to GM1, asialo GM1 and GM2. (Fukuta, et al, “Comparison of the Carbohydrate-binding Specificities of Cholera Toxin and Escherichia coli Heat-labile Enterotoxin LTh-1, LT-IIa and LT-IIb”, Infect. Immun. 56:1748-1753 (1988)) After binding of the B subunit to epithelial cell GM1 or GM2 receptors, the A1 subunit reaches the cytosol and after activation, binds to AND catalyzes ADP-ribosylation of Gsa.

Both CT and LT are immunogenic and nasal or oral exposure to either CT or LT results in secretory IgA (S-IgA) and serum antibodies, which are almost entirely restricted to anti-CT-B or anti-LT-B. Furthermore, both enterotoxins also act as strong mucosal adjuvants when co-administered with unrelated protein antigens, whether given by oral, nasal, or parenteral routes. It has also been shown that induction of maximal mucosal S-IgA and serum IgG antibody responses correlate directly with antigen-specific CD4⁺ T helper type 2 (Th2) cells secreting IL-4 and IL-5 in mice orally immunized with protein Ag and CT as adjuvant. (See Xu-Amano, et al., “Helper T Cell Subsets for IgA Responses: Oral Immunization with Tetanus Toxoid and Cholera Toxin as Adjuvant Selectively Induced Th2 cells in Mucosa Associated Tissues”, J. Exp. Med. 178:1309-1320 (1993).) Further, it has been shown that CT elicits adjuvant responses by inducing antigen-specific CD4⁺ Th2-type cells which produce high levels of IL-4 and IL-5, the cytokines which are responsible for supporting subsequent development of serum IgG1 and IgG2b subclass, IgE and mucosal S-IgA antibody responses. (Marinaro, et al, “Mucosal Adjuvant Effect of Cholera Toxin in Mice Results from Induction of T-helper 2 (Th2) Cells and IL-4:, J. Immunol. 155: 4621-4629 (1995)) However, oral immunization with LT promotes serum IgG2a with less IgG1 and IgG2b and good mucosal S-IgA antibody responses. This finding is supported by showing of a mixed CD4⁺ Th1- and IL-4-independent Th2-type response associated with production of the Th1-type cytokine IFN-γ as well as IL-5, IL-6 and IL-10 synthesis. (Takahashi, et al. “Mechanisms for Mucosal Immunogenicity and Adjuvancy of Escherichia coli Labile Enterotoxin”, J. Infect. Dis 173:627-635 (1996)) When IL-4 levels produced by antigen-specific CD4⁺ T cells were compared when either CT or LT were used as mucosal adjuvants, Ag-specific IL-4 production was much lower when LT was used. More recent work has shown that LT directly affects activated CD4⁺ T cells and inhibits IL-4 in normal mice and maintained IL-5 and IL-6 in IL-4 gene-deficient (IL-4^{-/-}) mice but CT preferentially inhibited Th1-type cytokines in normal mice and failed to support IL-5 and IL-6 in IL-4^{-/-} mice. These results clearly suggest that LT induces Th1-type as well as IL-4-independent Th2-type responses, while CT strongly supports Th2-type responses.

The use of viral and bacterial vectors as carriers for vaccines is well established. The most widely studied vectors are the pox viruses. The use of live vectors appears more likely to be of value in veterinary medicine than in immunization of humans. The use of such vectors presents several problems. It is difficult to regulate the live vectors. When attenuated organisms are used they may be susceptible to minor changes in the environment, such as pH in the gastrointestinal tract, which render them impotent as adjuvants. Furthermore, the microbial vectors may revert and become capable of causing disease in the host. Finally, viral and bacterial vectors are highly immunogenic and the host immune response to the vectors themselves limits their use to a single administration. It is necessary to find more dependable, less cumbersome adjuvants that will provide the benefits of directed immune response while avoiding the use of attenuated microbial vectors.

### BRIEF DESCRIPTION OF THE FIGURES

FIG. 1 shows the isotype of OVA-specific serum antibody responses, IgG subclass antibody responses and numbers of IgG AFC (antibody-forming cells) in cervical lymph nodes and spleen.

FIG. 2 shows OVA-specific IgA antibody responses in saliva and nasal washings and numbers of IgA AFC in nasal passages, lung and submandibular glands.

FIG. 3 shows OVA-specific CD4⁺ T cell proliferative responses.

FIG. 4 shows the amino acid and DNA sequence of the natural cholera toxin and the points at which mutations occurred.

### SUMMARY OF THE INVENTION

It is the purpose of this invention to provide customized adjuvants using chimeric mutants containing mutated A subunits of a first toxin coupled to B subunits from of a second toxin (an enterotoxin). The invention provides chimeric molecules comprising a first subunit which is mutated A subunit of a first enterotoxin and a second non-mutated B subunit from a second enterotoxin which is different from the natural enterotoxin which has been mutated to provide the A subunit. For example, similarities between CT, LT and other enterotoxins imply that B subunits can originate from other enterotoxins with the AB-type structure (i.e., vero toxin). Using methods of the invention, it is possible to customize adjuvants to direct production of cell-mediated or humoral immune responses. For example, the chimeric enterotoxins of the present invention retain the high adjuvanticity of native cholera toxin (CT) and the native toxin from which the B subunit is derived. However, the characteristics of the B subunit determines the nature of immune response which is induced to the co-administered protein.

### DETAILED DESCRIPTION OF THE INVENTION

The instant invention provides many of the benefits associated with use of microbial vectors as customized facilitators of immune response. The molecules made in accord with the methods of the invention provide means which are less cumbersome, less expensive and more reliable than the use of microbial vectors. These molecules provide much more predictable results than use of attenuated organisms.

The present invention arises from the discovery that though both A and B subunits of enterotoxins are required for adjuvant activity, it is the B subunit which determines whether the adjuvant will mainly induce cell-mediated (Th1-type) or humoral (Th2-type) immune responses. For example, CT-A/LT-B chimeras, like native LT, induce predominantly cell-mediated or Th1-type responses, while LT-A/CT-B chimeras, like native CT induce predominantly humoral or Th2-type responses. However, since both CT and LT induce diarrhea in humans, neither are suitable as mucosal adjuvants. The production of chimeras containing subunits from nontoxic CT mutants now make it possible to retain adjuvanticity and determine site at which immune response is effected. This discovery was made possible the construction and use of chimeric molecules containing a subunit from a mutant wherein the mutation of the A subunit has rendered the enterotoxin incapable of causing diarrhea but capable of initiating an immunological response and a B unit which is capable of directing the site of binding to direct response as it relates to Th1/Th2 type response. The chimeric molecules may be used to provide specific immune response to a particular enterotoxin (for example, CT-A/LT-B to provide immune response to LT) or may be used as adjuvants for use with unrelated vaccines.

It had previously been shown that nCT (natural cholera toxin) acts as a mucosal adjuvant by inducing CD4⁺ Th2-type cells which provide help for B cells making serum IgG1, IgG2b, IgA and IgE antibodies as well as mucosal S-IgA antibody responses. The possibility of obtaining more directed activity using methods of the invention has been exemplified in substitution of LT-B (for CT-B) in the cholera toxin molecule to create CT-A/LT-B chimeras which produce an enterotoxin with full mucosal adjuvant activity. More importantly, this CT-A/LT-B chimera induces serum IgG1 and IgG2a antibodies to co-mucosally administered proteins, and low serum IgE antibody responses wherein mucosal S-IgA antibodies are also maximally induced. Conversely, the LT-A/CT-B chimera induces serum antibody profile similar to nCT.

Analysis of antigen-specific CD4⁺ Th cells and derived cytokines showed that the CT-A/LT-B chimera, like native LT, induced Th1-type and IL-4-independent Th2-type responses. Native CT, native LT and CT-A/LT-B were assessed as nasal mucosal adjuvants in IL-4 gene knockout (IL-4^{-/-}) mice, a model previously shown to discriminate between nCT and nLT. While nCT fails to induce mucosal S-IgA antibodies in IL-4^{-/-} mice, LT induces brisk mucosal S-IgA antibodies to mucosally co-administered proteins. Because both CT-A/LT-B and nLT induced significant S-IgA antibody responses in IL-4^{-/-} mice, it was concluded that the A subunit is necessary for adjuvant activity. However, the B subunit actually directs the adjuvant-induced response through either Th1- or Th2-type pathways. The mutants of CT are non-toxic, but retain full mucosal adjuvant activity and induce CD4⁺ Th2-type responses.

Preparation of the Chimeric CT-A/LT-B Molecule

The chimeric enterotoxins were constructed as described below. CT-A was made by recombinant means and was also purchased from Sigma Chemical Co. (St. Louis, Mo.), CT-A was applied to an immobilized D-galactose column (Pierce Chemical Co., Rockford, Ill.) to remove any CT-B contamination. Eluted CT-A was found to contain no demonstrable B subunit, as shown by silver staining of the SDS-PAGE gel. LT-B was derived from an E. coli K12 strain DH5a transformant containing the plasmid (pJC217) that encodes the LT-B gene and was also purified by immobilized D-galactose chromatography. Purified LT-B was found to be structurally identical to the native B subunit, as determined by SDS-PAGE, and to contain no demonstrable A subunit, as shown by silver staining of the SDS-PAGE gel. LT-B and a slight excess of CT-A were mixed together, kept overnight at 40° C. and purified by gel filtration to obtain the associated chimeric CT-A/LT-B toxin. LT was purified by immobilized D-galactose chromatography using E. coli HB 101 strain transformant containing the plasmid (pKTN1003b) that encodes the LT gene as taught by Guidry, et al. (“Role of Receptor Binding in Toxicity, Immunogenicity and Adjuvanticity of Escherichia coli Heat-labile Enterotoxin”, Infect. Immun. 65:4943-4950 (1997)) and CT was purchased from List Biological Laboratories, Campbell, Calif. The chimeric LT-A/CT-B molecule was constructed using the same procedure described for the mirror CT-A/LT-B molecule by mixing LT-A and a slight excess of CT-B.

The sequence of the coding region and the amino acid sequence of nCT-A are well known. (See Nature, 306 (Dec. 8, 1983), pp551-556 and Biochimica et Biophysica Acta, 1090 (1991) pp. 129-141.) Two mutants of the CT-A were prepared. In the first, mutant CT (mCT) molecule was generated by single amino substitution, wherein glutamic acid (E) was substituted with lysine (K) at position 112 (E112K) in the A subunit. This required the replacement of TCC with TTT at the encoding nucleotide. The second mCT identified as S61F resulted from a mutation wherein, at amino acid 61, serine (S) was replaced with phenylalanine (F) resulting in the mutant identified as S61F. FIG. 4 shows the positions of the mutations to the sequence of the natural chain A of the cholera toxin.

Production of Recombinant CT-A (E112K) Mutant/LT-B Using a Bacillus brevis Expression System

The Bacillus brevis system represents an attractive expression system for production of recombinant proteins without LPS contamination associated with gram negative bacteria systems (i.e., E. coli). Thus the use of the Bacillus brevis system was used to produce nontoxic chimeric enterotoxins.

The recombinant mCTA E112K (or mCT-A)/LT-B chimera was generated by expressing a pNCMO2 plasmid containing the genes for LT-B and mCTA (pNCMO2-LTB-mCTA) into the Bacillus brevis HPD31 vector. The vector was cultured at 30° C. for 3 days and the mCT-A/LT-B in culture supernatant was purified using a D-galactose gel column (Pierce, Rockford, Ill.). The SDS page and Western blot analysis of the product confirmed the purity of this chimeric molecule and the existence of A and B subunits. Further, the toxicity assays that included the CHO assay, cAMP induction, and the ideal loop test confirmed the absence of enterotoxicity in this molecule.

Immunization Protocol Used With CT-A/LT-B Chimera

Mice were immunized intranasally with 100 μg of ovalbumin (OVA) (Sigma) together with 0.5 μg of chimera CT-A/LT-B, with 0.5 μg of nLT, or with 0.5 μg of nCT in PBS on days 0, 7 and 14.

Detection of Antigen-Specific Antibodies by ELISA and Antibody-Forming Cells (AFCs) by Enzyme-Linked Immunospot (ELISPOT) Assay

Antibody titers in serum and mucosal secretions were determined by ELISA by the method of Yamamoto, et al. (“A Nontoxic Mutant of Cholera Toxin Elicits Th2-Type Responses for Enhanced Mucosal Immunity”, Proc. Natl. Acad. Sci (USA) 94:5267-5272 (1997)). Endpoint titers were expressed as the reciprocal log₂ of the last dilution giving an optical density at 450 nm (OD₄₅₀) of ≧0.1 above negative controls. In the ELISPOT assay, antigen-specific AFCs from various tissues were determined by direct counting of spots.

The immune responses induced by mCT-A/LT-B as nasal adjuvant was also characterized to determine if the nontoxic chimera generated in the Bacillus brevis system retained its adjuvanticity and whether the nature of immune responses induced was also influenced by the origin of the B subunit.

Detection of Total and Antigen-Specific IgE in Serum

Total IgE levels were determined by ELISA as described by Yamamoto, et al. (1997). Antigen-specific serum IgE was detected by a modified IgE-capture luminometric assay. Endpoint titers were determined as the dilution of each sample showing a two-fold higher level of luminometric units above background.

OVA-Specific CD4⁺ T Cell Responses

CD4⁺ T cells from cervical lymph node cell suspensions were purified by use of the magnetic-activated cell sorter system in accord with the directions of the manufacturer (Miltenyi Biotec, Sunnyvale, Calif.). Cells were added to a nylon wool column (Polysciences, Warrington, Pa.) and incubated at 37° C. for 1 hour to remove adherent cells. The CD4⁺ T cell subset was then obtained by positive sorting using a magnetic bead separation system consisting of biotinylated anti-CD4 mAb (clone GK1.5) and streptavidin microbeads (MACS; Miltenyi Biotec). Purified splenic CD4⁺ T cells (>98% purity) were cultured at a density of 4×10⁶ cells/ml with OVA (1 mg/ml), T cell-depleted irradiated (3,000 rads) splenic feeder cells (8×10⁶ cells/ml) and IL-2 (10 units/ml) (PharMingen, San Diego, Calif.) in complete medium. The CD4⁺ T cell cultures were incubated for 4 days at 37° C. in 5% CO₂ in air. To measure cell proliferation, 0.5 μCi of [³H] thymidine (Du Pont/New England Nuclear Products, Boston, Mass.) was added to individual culture wells 18 hours before termination, and the uptake of counts per minute by dividing cells was determined by scintillation counting.

Cytokine Analysis by ELISA

Cytokine levels in culture supernatants were determined by a cytokine-specific ELISA. Nunc MaxiSorp Immunoplates (Nunc, Naperville, Ill.) were coated with monoclonal anti-mouse IFN-γ, IL-2, IL-4, IL-5, IL-6 and IL-10 antibodies (PharMingen). After blocking, cytokine standards (PharMingen) and serial dilutions of culture supernatants were added in duplicate. For secondary antibodies and detection enzymes, biotinylated rat anti-mouse cytokine mAb (PharMingen) and peroxidase-labeled anti-biotin antibodies (Vector Laboratories) were employed and developed with TMB containing H₂O₂ (Sigma).

RT-PCR Analysis of Cytokine-Specific mRNA

For detection of cytokine-specific mRNA (IFN-γ, IL-4 and IL-10) in CD4⁺ T cells a standard reverse transcriptase PCR (RT-PCR) amplification protocol was used. Total RNA fractions were prepared from antigen-stimulated CD4⁺ T cells by the acid guanidinium-thiocyanate, phenol-chloroform extraction method. Quantitative cytokine-specific RT-PCR was performed by modification of the methods of Hiroi, et al. (“Polarized Th2 Cytokine Expression by both Mucosal Gamma Delta and Alpha Beta T cells” Eur. J. Immunol. 25:2743-2751 (1995)).

Adjuvant Activity and Properties of CT-A/LT-B

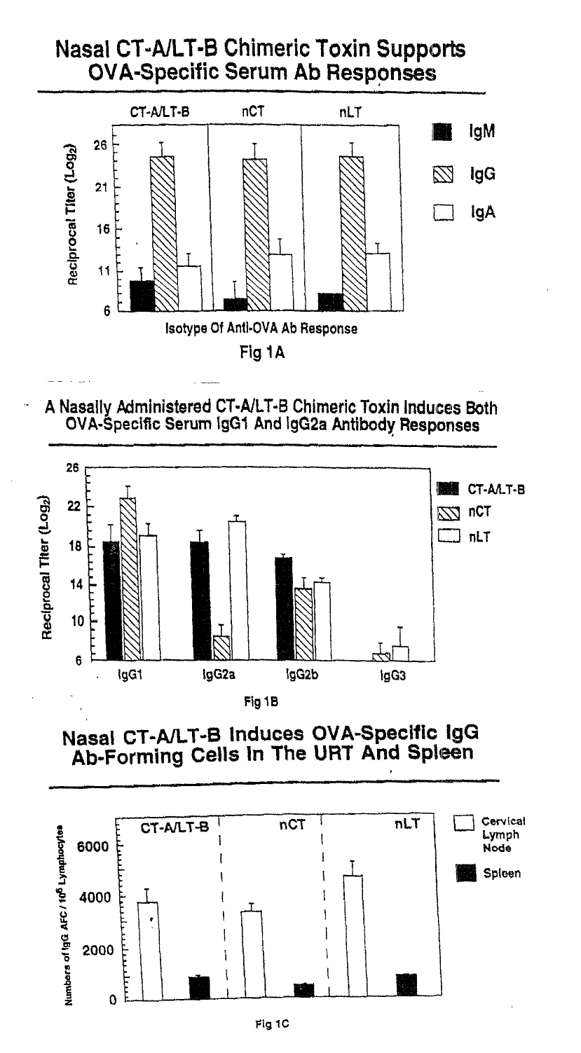

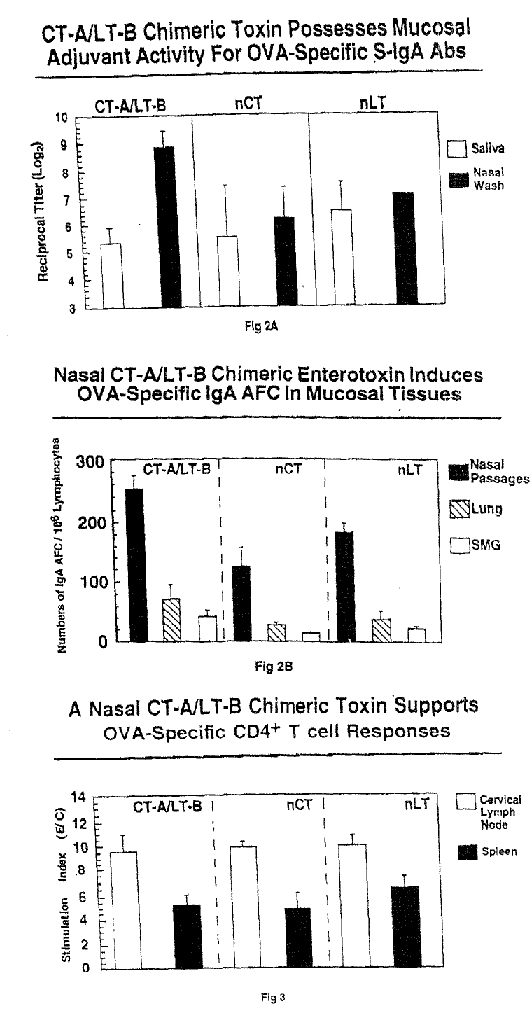

In the initial studies, the chimera CT-A/LT-B was assessed for use as a potential mucosal adjuvant by examining systemic and mucosal antibody responses of mice given OVA and adjuvant by the nasal route. Mice nasally immunized with OVA plus the chimeric toxin showed comparable serum antibody titers of OVA-specific IgM, IgG and IgA isotypes as those induced by nCT or nLT as an adjuvants. Interestingly, analysis of IgG subclass responses in mice given OVA plus the chimera toxin revealed that the major subclasses were IgG2a, with less IgG1 and less IgG2b and were remarkably similar to those seen when nLT was given as an adjuvant. In contrast, nCT mainly induced IgG1 and IgG2b antibody responses. Examination of OVA-specific IgG secreting cells in cervical lymph nodes and spleen showed that nearly equivalent numbers were induced by each adjuvant. (See FIG. 1, which shows the isotype of OVA-specific serum antibody responses (A), IgG subclass antibody responses (B) and numbers of IgG AFC in the cervical lymph nodes (CLN) and spleen (C). Groups of C57BL/6 mice were nasally immunized with 100 μg of the protein ovalbumin (OVA) plus 0.5 μg of the chimera CT-A/LT-B, 0.5 μg of native CT, or 0.5 μg of native LT on days 0, 7 and 14 and the isotypes of serum Ab titers were assessed on day 21. Mononuclear cells were isolated from CLN and spleen and were then examined for the isotype of AFC by ELISPOT. The results are expressed as the mean ± SEM obtained for five mice per group in 4 separate experiments.) Nasal co-administration of the chimera toxin induced OVA-specific mucosal IgA antibody responses in saliva and nasal wash samples. The responses in saliva were comparable to those induced by nLT or nCT as an adjuvant and the responses in nasal wash were slightly higher than those induced by nLT or nCT. Assessment of the numbers of anti-OVA IgA AFCs in several mucosal tissues revealed that when mice were given OVA plus the chimeric toxin there was a significant increase in the numbers of OVA-specific AFC when compared with those generated by nCT or nLT, including cells derived from the nasal passages (See FIG. 2, which shows OVA-specific IgA antibody responses in saliva and nasal washes (A) and the numbers of IgA AFC in nasal passages, lung and submandibular glands (SMG) (B). Groups of C57BL/6 mice were immunized with 100 μg of OVA plus 0.5 μg of chimera CT-A/LT-B, 0.5 μg of nCT or 0.5 μg of nLT as shown in FIG. 1. Tissue samples were assessed for IgA AFC and external secretions for IgA antibody titers at day 21. The results are expressed as the mean ± SEM obtained for five mice per group in 4 separate experiments.) The numbers of OVA-specific AFCs correlated with the serum and mucosal S-IgA antibody titers which were observed when mice were given OVA plus the chimera toxin, nLT or nCT as mucosal adjuvant.

Serum IgE Responses

Serum IgE responses induced by nasal immunization with OVA plus the chimeric toxin, nLT or nCT as adjuvant revealed that the chimeric toxin induced slightly higher levels of IgE when compared to nLT and the titer was less than that induced by nCT. On the other hand, mice which lack the IL-4 gene showed undetectable IgE synthesis with all of the adjuvants employed, indicating the involvement of IL-4 and the Th2-type pathway in enhancing OVA-specific IgE antibody responses by the chimeric toxin as well as nCT and nLT.

OVA-Specific CD4⁺ T Cell Responses

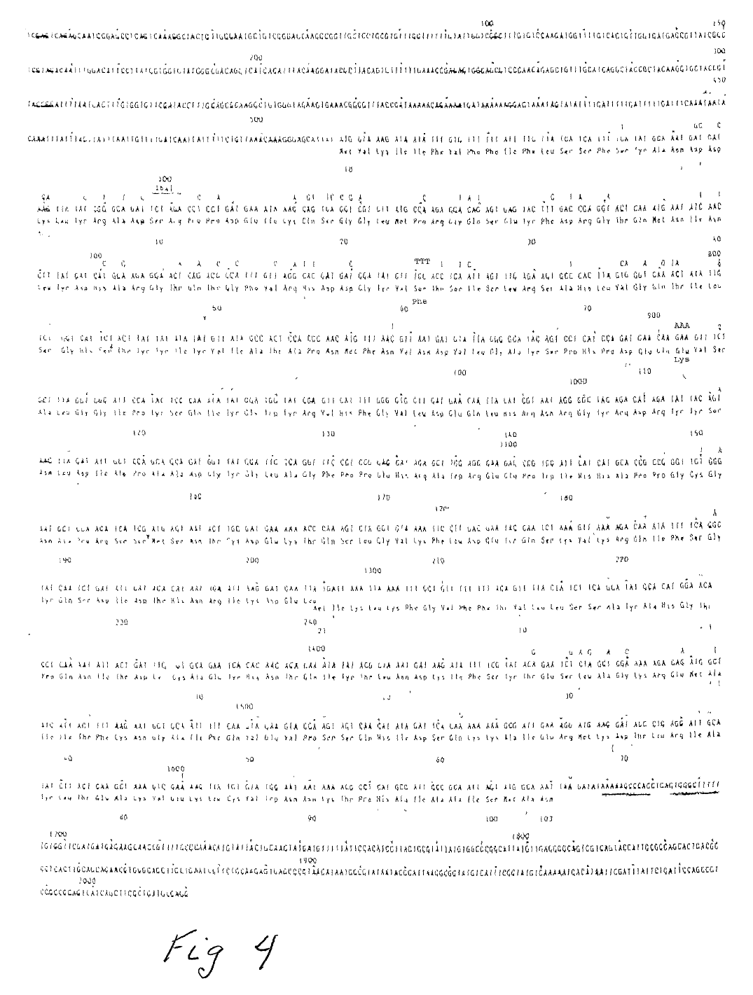

Significant and similar levels of proliferative responses by CD4⁺ T cells from CLN were observed when the chimeric toxin, nLT or nCT were used as mucosal adjuvants. (See FIG. 3, which shows OVA-specific CD4⁺ T cell proliferative responses. Groups of C57BL/6 mice were immunized with 100 μg of OVA plus 0.5 μg of chimera CT-A/LT-B, 0.5 μg of nCT or 0.5 μg of nLT as seen in FIG. 1. CD4⁺ T cells were isolated from CLN or spleen on day 21 and were cultured with 1 μg/ml of OVA in the presence of irradiated, T cell-depleted spleen cells as feeder cells and recombinant IL-2. The results are representative of three separate experiments.) Cytokine analysis at the mRNA level showed that OVA-specific CD4⁺ T cells from CLN of mice given OVA plus the chimeric toxin produced both IFN-γ and IL-2, (Th1) as well as low IL-4 and higher IL-5, IL-6, and IL-10 (Th2) specific mRNA, indicating that a Th1- and an IL-4-independent Th2-type helper T cell response was induced in both mucosal and systemic tissues when mice were immunized intranasally with the chimeric toxin as mucosal adjuvant. A similar pattern was seen when nLT was used as adjuvant. When IL-4^{-/-} mice were given OVA plus the chimeric toxin, the cytokine-specific mRNA pattern did not change other than the fact that IL-4 was not detected, indicating the involvement of a major Th1 and IL-4-independent Th2 T cell response to antigen with the chimeric toxin as well as with nLT. At the protein level, significantly increased levels of IFN-γ production, which greatly exceeded the levels induced by nCT but which were slightly less than induced by nLT, were seen in OVA-specific CLN cultures from mice nasally immunized with OVA plus the chimeric toxin. On the other hand, IL-4 production was highest in OVA-specific T cell cultures from mice nasally immunized with OVA plus nCT as mucosal adjuvant. The data support the results of cytokine synthesis at the mRNA level. IFN-γ production in CLN from IL-4^{+/-} mice immunized with OVA plus nCT was significantly increased in contrast to that from IL-4^{-/-} mice immunized with OVA plus nCT. Unlike the case of nCT, the increases in IFN-γ, when the chimeric toxin was co-administered to IL4^{+/+} mice, were modest and levels were almost identical to those of the OVA plus nLT group.

While the results described above clearly show that the CT-A/LT-B chimera promotes immune responses resembling those induced by nLT, it was also found that the mirror molecule LT-A/CT-B induces immune responses resembling those mediated by nCT.

Summarizing symptoms arising from the antigenic molecules:

| Antigen | | physical responses |
|---|---|---|
| nCT or nLT | → | both are toxic, induce diarrhea |
| Mutant CTs | → | both are non-toxic and non- |
| (S61F or E112K) | | diarrheagenic |
| CT-A (S61F or E112K) | → | non-toxic, non-diarrheagenic |
| plus CT-B | | |
| CT-A (S61F or E112K) | → | non-toxic, nondiarrheagenic |
| plus LT-B | | |

Summarizing the unique properties of chimeric enterotoxin

| Antigiens/Adjuvants | | CD4⁺ Th1/Th2 | Antibodies |
|---|---|---|---|
| native CT, mCT, | → | CD4⁺ Th2⁺ | serum IgG1, IgA |
| and | | | IgE and mucosal |
| S61F/E12K (CT-A/CT-B) | | | |
| S-IgA | | | |
| Native LT | → | Th1-, Il-4- | serum IgG2a, IgA |
| and | | negative Th2⁺ | low IgE and |
| mucosal | | | S-IgA |
| CT-A/LT-B | → | Th1⁺, IL-4- | serum IgG2a, IgA |
| and | | negative Th2⁺ | low IgE and |
| mucosal | | | S-IgA |
| LT-A/CT-B | → | Th2⁺ | serum IgG1, IgA |
| and | | | IgE and mucosal |
| S-IgA | | | |

Statistical Analysis

Results are reported as the mean ± one standard error (SE). Statistical significance (p <0.05) was determined by the Student's t test and by the Mann-Whitney U test of unpaired samples.

In view of the above, it is clear that it is now possible, using the method of the invention, to use non-toxic chimeric enterotoxins which provide site-directed immune response, thus designing design vaccine compositions to selectively produce humoral and/or cellular antibodies.

Examples of human vaccines containing immunogenic antigens are found, for example, in the Mayo Clinic Family Health Book D. Larson, M.D., Ed., William Marrow & Co., Inc. (1990) Methods of preparing vaccines are well known in the art, as seen, for example, in U.S. Pat. No. 5,419,907, which is incorporated herein by reference in its entirety. Compositions containing the chimeras of the invention may, additionally, contain other adjuvants and diluents known in the art.

The compositions containing the chimeric constructs may be given systemically or administered directly to the mucosa of the gastrointestinal or respiratory tract. Adjuvants of the invention may, for example, be administered orally, nasally, subcutaneously, intracutaneously, intramuscularly or dermally by patch. They may be administered with other immunogens, whether natural or mutated.

The form of the composition will depend on the method of administration. Both liquid and capsular compositions may be appropriate for administration orally. For nasal administration, liquids or powders (for example, lyophilized chimeric proteins) may be administered. The constructs of the invention may also be provided on solid supports.

What we claim is:

1. A method of providing directed immune response comprising production of a construct having a non-toxic mutated A subunit from a first enterotoxin and a B subunit from a second enterotoxin wherein the B subunit is chosen to direct the site of binding.

2. The method of claim 1 wherein the B subunit is chosen to induce cell-mediated immune response.

3. The method of claim 1 wherein the B subunit is chosen to induce humoral immune response.

4. A chimeric molecule comprising a first subunit which is mutated A subunit of a first enterotoxin and a second non-mutated subunit from a second enterotoxin which is different from the natural enterotoxin which has been mutated to provide the A subunit.

5. A composition of matter comprising chimeric molecules (chimeras) of claim 4 in a pharmaceutically acceptable carrier.

6. A method of obtaining enhanced immune response of an organism to an antigen by administration of said antigen with a composition of claim 5.

7. A chimeric molecule of claim 4 wherein the first subunit which is mutated A subunit of cholera toxin wherein the serine (amino acid 61) of the natural toxin has been replaced by a phenylalanine in the first enterotoxin and the second non-mutated subunit from a second enterotoxin the B chain of labile toxin of E. coli.

8. A chimeric molecule of claim 4 wherein the first subunit which is mutated A subunit of cholera toxin wherein the glutamine (amino acid 112) of the natural toxin has been replaced by a lysine in the first enterotoxin and the second non-mutated subunit from a second enterotoxin the B chain of labile toxin of E. coli.

9. A composition of matter comprising a chimeric molecule of claim 7 in a pharmaceutically acceptable carrier.

10. A composition of claim 9 wherein, in the chimeric molecule, the first subunit which is mutated A subunit of cholera toxin wherein the glutamine (amino acid 112) of the natural toxin has been replaced by a lysine in the first enterotoxin and the second non-mutated subunit from a second enterotoxin the B chain of labile toxin of E. coli.

11. A composion of claim 9 whereif, in the chimeric molecule, the first subunit which is mutated A subunit of cholera toxin wherein the glutamine (amino acid 112) of the natural toxin has been replaced by a lysine in the first enterotoxin and the second non-mutated subunit from a second enterotoxin the B chain of labile toxin of E. coli.
